(19) United States
(12) Patent Application Publication
(10) Pub. No.: US 2012/0089212 A1
(43) Pub. Date: Apr. 12, 2012

(54) Thermal Pad

(76) Inventors: Steven J. Benda, Cokato, MN (US); Carla A. Hendrickson, Cokato, MN (US)

(21) Appl. No.: 13/270,519

(22) Filed: Oct. 11, 2011

Related U.S. Application Data
(60) Provisional application No. 61/404,966, filed on Oct. 12, 2010.

(51) Int. Cl. A61F 7/00

(52) U.S. Cl. 607/108; 607/114

(57) ABSTRACT

Some embodiments provide a thermal pad having a sealed outer pouch and a sealed inner pouch within the outer pouch. A thermally transmitting material is encased by the inner pouch for transmitting heat or cold to a user of the thermal pad. In some cases the thermal pad also includes a baffling mechanism that divides the interior of the inner pouch into at least two areas and is configured to permit manual manipulation of the thermally transmitting material between the at least two areas and to at least partially retain the material within one or more of the at least two areas subsequent to manual manipulation. In some cases the thermal pad includes first and second layers of thermal insulation positioned between the inner pouch and outer pouch. Methods for administering thermal therapy to a woman's vaginal area are also provided.

### CROSS-REFERENCES

This application claims the benefit of U.S. Provisional Application No. 61/404,966, filed Oct. 12, 2010, the content of which is hereby incorporated by reference in its entirety.

### FIELD

This disclosure relates to a thermal pad for administering thermal therapy to a user. In particular, the disclosure pertains to a thermal pad configured for administering thermal therapy to a woman's vaginal area.

### BACKGROUND

Vulvar varicosity is a poorly publicized fact of life for many women, particularly, during pregnancy. This condition is often referred to as “Vaginal Varicose Veins” and it is a swollen or a dilated condition of the veins in the vulvar portion of the vagina. Veins are the pathway for blood to return to the heart and contain valves that close to prevent backflow of blood between heartbeats due to the effects of gravity and increased blood volume. Weakening of these vein valves along with a woman's increased blood volume during pregnancy often causes blood to stagnate in the veins resulting in inflammation of the veins or what is called phlebitis. The regions of stagnated blood will appear as soft, swollen, dark blue bulges and these areas are often itchy and painful. Generally, the remedy for minimizing or eliminating the pain and discomfort caused by vaginal varicose veins include one or more of: dietary alterations and/or enhancements, support or compressive tights or garments, application of bulky ice packs or frozen peas, application of distilled witch hazel, application of alternate hot and cold shower spray to the affected areas, etc. However, such methods and techniques provide only a modicum level of relief to a pregnant woman during these phlebitis conditions.

### SUMMARY

According to one aspect of the invention, a thermal pad is provided. The thermal pad includes a sealed, elongated outer pouch and a sealed, elongated inner pouch within the outer pouch. The outer pouch has a wall formed from an impermeable, pliant first material and the inner pouch has a wall formed form an impermeable, pliant second material. The wall of the outer pouch includes a first section having an exterior surface for applying to a treatment area and a second section having an exterior surface that faces away from the exterior surface of the first section. The thermal pad also includes a viscous thermally transmitting material encased by the inner pouch for transmitting heat or cold to a user of the thermal pad. The thermal pad may also include a baffling mechanism positioned within the inner pouch. The baffling mechanism divides the interior of the inner pouch into at least two areas having fluid communication therebetween. The baffling mechanism is also configured to permit manual manipulation of the thermally transmitting material between the at least two areas and to at least partially retain the thermally transmitting material within one or more of the at least two areas subsequent to manual manipulation.

According to another aspect of the invention, a thermal pad is provided. The thermal pad includes a sealed, elongated outer pouch and a sealed, elongated inner pouch within the outer pouch. The outer pouch has a wall formed from an impermeable, pliant first material and the inner pouch has a wall formed form an impermeable, pliant second material. The wall of the outer pouch includes a first section having an exterior surface for applying to a treatment area and a second section having an exterior surface that faces away from the exterior surface of the first section. The thermal pad also includes a viscous thermally transmitting material encased by the inner pouch for transmitting heat or cold to a user of the thermal pad. The thermal pad also includes first and second layers of thermal insulation. The first layer of thermal insulation is positioned between the first section of the outer pouch wall and the inner pouch, while the second layer of thermal insulation is positioned between the second section of the outer pouch wall and the inner pouch.

According to another aspect of the invention, a method is provided for administering thermal therapy to a woman's vaginal area. The method includes configuring a thermal pad in a first treatment configuration, cooling or heating a thermally transmitting material within the thermal pad, and positioning the thermal pad adjacent to a woman's vaginal area to allow heat transfer between the vaginal area and the thermally transmitting material. The thermal pad includes a sealed elongated outer pouch, a sealed elongated inner pouch within the outer pouch, the viscous thermally transmitting material encased by the inner pouch, and a baffling mechanism positioned within the inner pouch. Configuring the thermal pad in a first treatment configuration includes manipulating the thermal pad to direct the viscous thermally transmitting material about the baffling mechanism in a desired distribution.

Embodiments of the present invention can provide one or more of the following features and/or advantages.

In some cases, the first and second sections of the outer pouch include opposing first and second layers of the first material sealed along at least a portion of a periphery of the outer pouch. Similarly, the inner pouch may optionally include opposing first and second layers of the second material, sealed along at least a portion of a periphery of the inner pouch. In some cases a layer of thermal insulation is included between the first section of the outer pouch wall and the inner pouch and another layer of thermal insulation is positioned between the second section of the outer pouch wall and the inner pouch. In some cases the thermal resistance of the first layer of thermal insulation is less than a thermal resistance of the second layer of thermal insulation to promote greater heat transfer through the first insulation layer (e.g., to the treatment area) than through the second insulation layer (e.g., to the external environment).

In some cases a thermal pad includes a baffling mechanism that is configured to restrict flow of the thermally transmitting material within the inner pouch. The baffling mechanism divides the interior of the inner pouch into multiple (e.g., two or more) areas having fluid communication therebetween. In such a case, the baffling mechanism restricts flow of the thermally transmitting material between two or more of the areas of the inner pouch interior. In some cases the inner pouch has a longitudinal axis, and four areas of the interior of the inner pouch include front and back areas proximate opposite ends of the longitudinal axis and first and second side areas on opposite sides of the longitudinal axis between the front and back areas. In some cases the baffling mechanism is configured to restrict flow of the thermally transmitting material in at least two directions within the inner pouch. The baffling mechanism can optionally include one or multiple baffles that restrict fluid flow within the inner pouch. In some cases the baffling mechanism includes a longitudinal member positioned along a length of the interior of the elongated inner pouch and at least one lateral member positioned across the longitudinal member.

In some cases the thermal pad is configured for administrating thermal therapy to a woman's vaginal area. The first and second materials of the inner and outer pouch walls are optionally pliant, which with the viscous nature of the thermally transmitting material, enable the pouch walls to form a specific geometrical surface configuration in response to pressure applied by the shape and varying contours of a woman's vulvar area. In some cases the thermal pad is configured to substantially cover an anterior section, a central section, and a posterior section of the vaginal area. When positioning the thermal pad adjacent a woman's vaginal area, a first layer of thermal insulation can be in closer proximity to the vaginal area than a second layer of thermal insulation having a higher thermal resistance than the first layer of insulation. Thus, the first layer of insulation can tend to enable an amount of heat transfer from the thermally transmitting material to the vaginal area and the second layer of insulation can tend to inhibit heat transfer from the thermally transmitting material to the exterior environment.

These and various other features and advantages will be apparent from a reading of the following detailed description.

### BRIEF DESCRIPTION OF THE DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIG. 1 is a top plan view of the thermal pad in accordance with an embodiment of the invention;

FIG. 2 is a bottom plan view of the thermal pad of FIG. 1;

FIG. 3 is a cross-sectional view from approximately a center of a thermal pad in accordance with an embodiment of the invention;

FIG. 4 is a cross-sectional view of a section towards the back of a thermal pad in accordance with an embodiment of the invention;

FIG. 5 is an illustration of pre-positioning a thermal pad relative to a treatment area in accordance with an embodiment; and

FIG. 6 is an illustration of the thermal pad of claim 5 in position against the treatment area.

### DETAILED DESCRIPTION OF THE PREFERRED EMBODIMENTS

The following detailed description is exemplary in nature and is not intended to limit the scope, applicability, or configuration of the invention in any way. Rather, the following description provides some practical illustrations for implementing exemplary embodiments of the present invention. Examples of constructions, materials, dimensions, and manufacturing processes are provided for selected elements, and all other elements employ that which is known to those of ordinary skill in the field of the invention. Those skilled in the art will recognize that many of the noted examples have a variety of suitable alternatives.

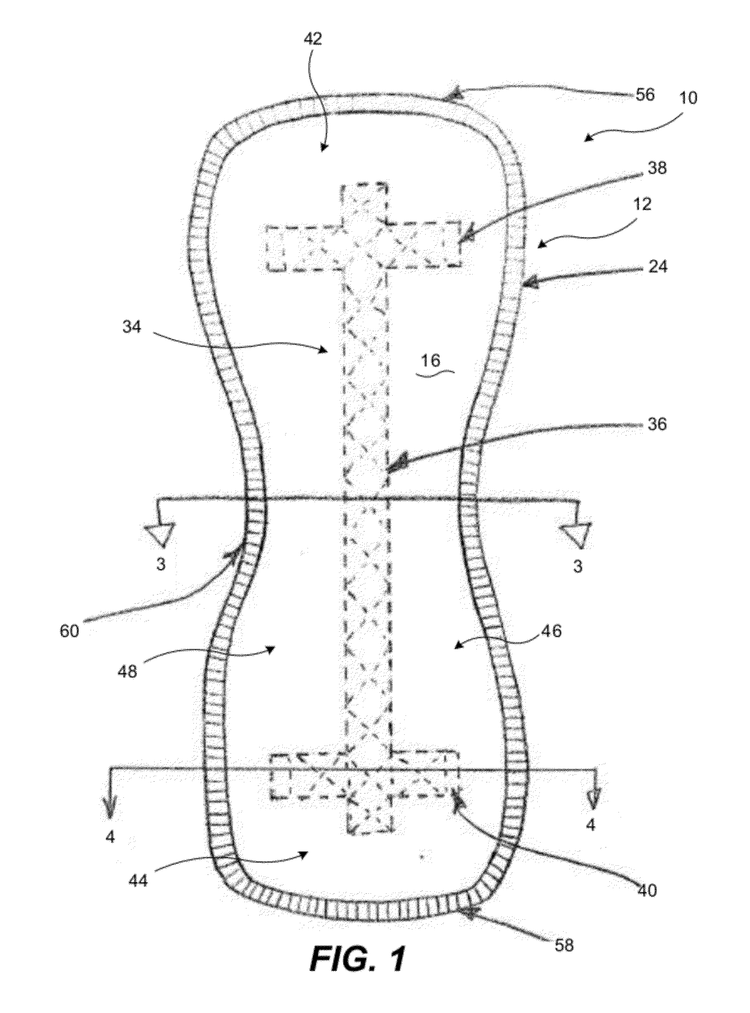

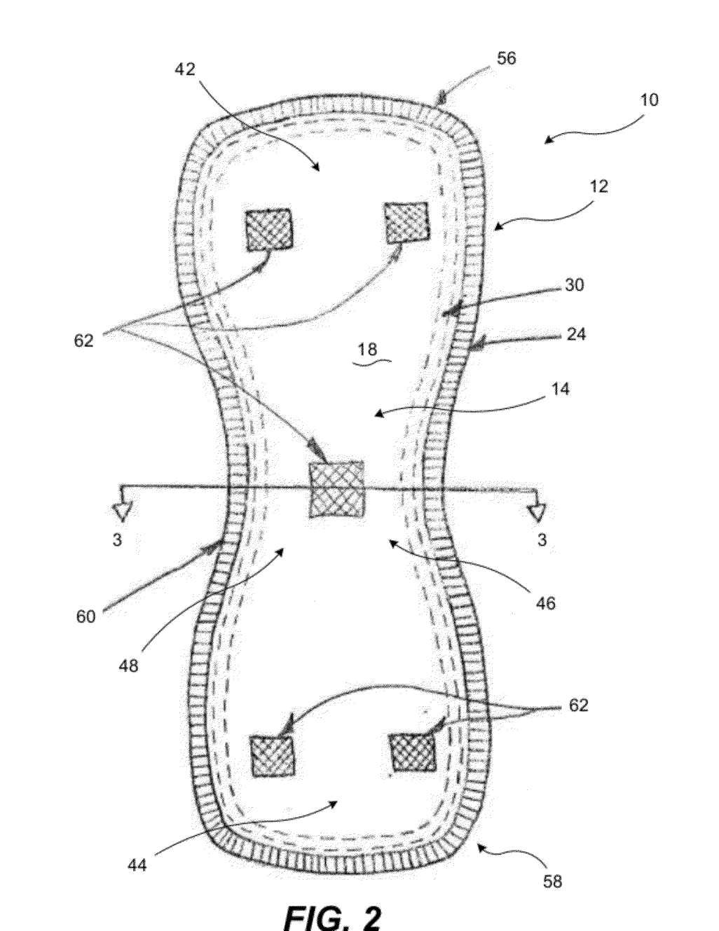

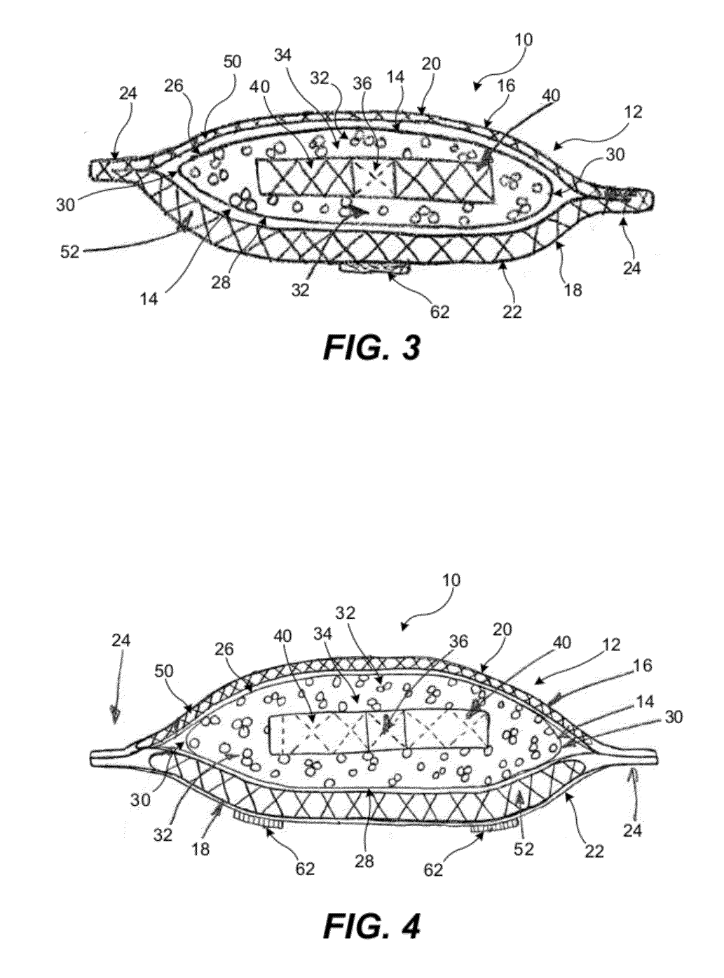

FIGS. 1-4 show various views of a thermal pad 10 in accordance with non-limiting exemplary embodiments of the invention. FIGS. 1 and 2, respectively, are top and bottom plan views, and FIGS. 3 and 4, respectively, are cross-sectional views proximate a center region of thermal pad 10 and proximate a back region of the thermal pad 10.

Thermal pad 10 includes sealed elongated outer pouch 12 and sealed elongated inner pouch 14 contained within outer pouch 12. The walls of outer pouch 12 and inner pouch 14, respectively, are formed from one or more impermeable pliant materials. In some cases, the wall of the outer pouch 12 is formed from a first material and the wall of the inner pouch 14 is formed from a second material, which may or may not be the same as the first material. The wall of outer pouch 12 includes a first section having exterior surface 16 for application against a treatment area, and a second section having exterior surface 18 facing away from exterior surface 16. The first and second sections of outer pouch 12 respectively include opposing first and second layers 20 and 22 of the first material.

In some cases, first and second layers 20 and 22 are defined by a single sheet of the first material sealed along at least a portion of periphery 24 of outer pouch 12. Optionally, the first and second layers 20 and 22 can be defined by separate sheets of the first material having their respective perimeters sealed along at least a portion of periphery 24 of outer pouch 12. In some cases, outer pouch 12 is an extrusion formed with the first material. Similarly, first and second layers 26 and 28 of inner pouch 14 are defined by either a single sheet or separate sheets of the second material sealed along at least a portion of periphery 30 of inner pouch 14. Alternatively, inner pouch 14 is an extrusion formed with the second material.

The inner and outer pouches 14, 12 can be formed from any suitable material. In some cases the first material (and optionally the second material) is non-absorbent and washable so that thermal pad 10 can be cleaned after use and then reused. In some embodiments, one or both of the first and second material is a plastic sheathing or a thermoplastic film or a sealable polymer film. The first material may be the same as the second material, or may optionally be different than the second material.

Thermal pad 10 further comprises viscous thermally transmitting material 32 encased by the impermeable pliant material of inner pouch 14. Thermally transmitting material 32 is a material that can be heated or cooled prior to the placement of exterior surface 16 against the treatment area. Once applied to the treatment area, thermal pad 10 transmits heat or cold from the thermally transmitting material 32 through the inner pouch wall, through the outer pouch wall and to the treatment area. In an embodiment of the invention, thermally transmitting material 32 is a viscous gelatinous material that tends to retain the presence or absence of heat for a period of time. Alternate material suitable for use as thermally transmitting material 32 will be apparent to one skilled in the art. All such alternatives are considered as being within the intent, scope and spirit of the present invention.

In a non-limiting exemplary embodiment, thermal pad 10 comprises baffling mechanism 34 positioned within inner pouch 14. Baffling mechanism 34 is configured such that it divides the interior of inner pouch 14 into two or more areas, enables fluid communication of thermally transmitting material 32 between the two or more areas, and thereafter at least partially retains thermally transmitting material 32 in the two or more areas as distributed due to the viscous nature of thermally transmitting material 32 and the structure of the baffling mechanism 34. Accordingly, different quantities (or proportions) of thermally transmitting material 32 can be distributed by manual manipulation into the different areas of pad 10 as desired.

As shown in FIG. 1, in some cases a baffling mechanism 34 includes longitudinal member 36 positioned substantially along the length and longitudinal axis of elongated inner pouch 14 and two lateral members 38 and 40 positioned across and proximate the opposite ends of longitudinal member 36. In this case baffling mechanism 34 divides the interior of inner pouch 14 into at least four areas with fluid communication therebetween. These four areas are identified as front and back areas 42 and 44 respectively located proximate opposite ends of longitudinal member 36, and side areas 46 and 48 on opposite sides of the longitudinal axis between front and back areas 42 and 44. Baffling mechanism 34 enables manual manipulation of thermally transmitting material 32 into and out of the front, back and the two side areas 42, 44, 46 and 48, respectively, and thereafter at least partially retains thermally transmitting material 32 in the areas as distributed by the manual manipulation.

In some cases the baffling mechanism includes elongated members such as those shown in FIG. 1. It is contemplated that the baffle members can be provided in a number of shapes and sizes that provide fluid communication between adjacent areas and allow manual manipulation of thermally transmitting material between areas while at least partially retaining the thermally transmitting material within the areas to which is has been distributed. In some cases the baffling mechanism may extend substantially between the first and second layers 26 and 28 of inner pouch 14, but allow movement of thermally transmitting material 32 between the baffling mechanism and one or both of first and second layers 26, 28 by manual squeezing or other manipulation. In some cases the baffling mechanism 34 may optionally be positioned between the layers with an amount of space between one or both layers and the baffling mechanism 34 as shown in FIGS. 3 and 4. In some cases it is contemplated that baffling mechanism 34 may include a plurality of baffles, walls, openings, and/or valves (not shown) that are configured to allow distribution and retention of thermally transmitting material 32 in the areas within inner pouch 14.

In some cases baffling mechanism 34 is formed from flexible rubber, dense gauze material, 3D forms of the shell pliable material or other non-degrading materials that can be permanently located in the thermally transmitting material 32. In addition, the baffling mechanism may be suspended within the thermally transmitting material 32 and move somewhat as the material is manipulated around the baffling mechanism. In some cases the baffling mechanism could instead be attached to one or more of the inside surfaces of the first pouch 14, or the inner pouch 14 may be stretched somewhat about the baffling mechanism 34 to maintain it in place.

In some cases, baffling mechanism 34 comprises more or less lateral members similar to lateral members 38 and 40, and accordingly divides the interior of inner pouch 14 into more than or less than the four areas described above. For instance, in an embodiment of baffling member 34 with no lateral members, the interior of inner pouch 14 is divided into two areas 46 and 48 extending along the length of thermal pad 10 on opposite sides of the longitudinal axis. In another embodiment of baffling member 34 comprising one of the two lateral members, for example lateral member 38, the interior of inner pouch 14 is divided into three areas identified as front area 42 and areas 46 and 48 extending from front area 42 along the length of thermal pad 10 on opposite sides of the longitudinal axis. As will be apparent to one skilled in the art, the one or more lateral members can be positioned anywhere along the length of longitudinal member 36. Additionally, while lateral members 38 and 40 are illustrated as being generally straight and orthogonal to longitudinal member 36, it will be apparent to one skilled in the art that each one of the one or more lateral members can be at any desired geometric angle with respect to the longitudinal member 36 and/or may have curves or another desired geometry. In addition, it should be appreciated that in some embodiments of the invention, thermal pad 10 does not include baffling mechanism 34.

As previously stated, thermal pad 10 is configured for transmitting heat or cold between thermally transmitting material 32 and the treatment area against which exterior surface 16 is placed. In order to assist in the efficient transmission of heat or cold to the treatment area and to reduce thermal losses, some embodiments of thermal pad 10 comprise one or more layers of thermal insulation between outer pouch 12 and inner pouch 14. As illustrated in FIGS. 3 and 4, thermal pad 10 includes first layer of thermal insulation 50 between the first section of the wall of outer pouch 12 and the opposing wall of inner pouch 14, and second layer of thermal insulation 52 between the second section of the wall of outer pouch 12 and the opposing wall of inner pouch 14. The insulation layers 50, 52 can be formed from any suitable type of material exhibiting a desired degree of thermal insulation. As just one example, padding material such as that found in panty liners may be used.

In some embodiments, the thermal resistance of first layer of thermal insulation 50 is less than the thermal resistance of second layer of thermal insulation 52, whereby the heat transfer between thermally transmitting material 32 and the treatment area will be greater than the heat transfer between thermally transmitting material 32 and an external area adjacent to the exterior surface of the second section of the wall of outer pouch 12. In other embodiments, both first and second layers of thermal insulation 50 and 52, respectively, have substantially equal thermal resistances. Differences in thermal resistance can be accomplished through the use of different materials for each of the layers of insulation 50, 52, or by using differing amounts of thermal insulation for each layer.

Turning to FIG. 3, in at least one non-limiting exemplary embodiment of the invention, first and second layers of thermal insulation 50 and 52, respectively, extend into the seal along at least a portion of periphery 24 of outer pouch 12. Turning to FIG. 4, in some cases first and second layers of thermal insulation 50 and 52, respectively, extend proximate to but not into the seal along at least a portion of periphery 24 of outer pouch 12. Of course the extent (e.g., surface area) of each insulation layer can vary and may not be the same for each layer.

FIGS. 3 and 4 illustrate an embodiment of thermal pad 10 including a first gap between the outside surface of the wall of inner pouch 14 and inside surfaces of both first and second layers of thermal insulation 50 and 52. As such, outside surfaces of both first and second layers of thermal insulation 50 and 52 are respectively shown as being substantially adjacent to or in contact with the inside surfaces of first and second layers 20 and 22 (or the inside surface of the wall of outer pouch 12). In alternate embodiments, thermal pad 10 includes no gaps or includes one or more gaps between the walls of outer and inner pouches 12 and 14 and one or both first and second layers of thermal insulation 50 and 52. For instance, an embodiment of thermal pad 10 does not include the first gap as described herein above and, as such, both first and second layers of thermal insulation 50 and 52 are respectively sandwiched between the walls of outer and inner pouches 12 and 14. Accordingly, the outside surfaces of both first and second layers of thermal insulation 50 and 52 are respectively substantially adjacent to or in contact with the inside surfaces of the wall of outer pouch 12 (or inside surfaces of first and second layers 20 and 22). Similarly, the inside surfaces of both first and second layers of thermal insulation 50 and 52 are respectively substantially adjacent to or in contact with the outside surfaces of the wall of inner pouch 14. In another embodiment, thermal pad 10 includes the first gap as described herein above and further includes a second gap between the inside surface of the wall of outer pouch 12 and the outside surfaces of both first and second layers of thermal insulation 50 and 52. In yet another embodiment, thermal pad 10 includes the second gap but does not include the first gap.

As discussed above, in some cases the first and/or second layers of insulation 50, 52 may be secured between the inner and outer pouches 14, 12 by capturing an edge of the insulation layer between a sealed portion of the periphery of the outer pouch 12. In some cases one or both insulation layers 50, 52 may be adhered to one or both of the walls of the inner and outer pouches 14, 12. In some cases the first and/or second layers of insulation 50, 52 may be simply positioned loosely between the inner and outer pouches without any additional securing mechanism. Further alternative embodiments, for example thermal pad 10 with no layers of thermal insulation or with one, two, or more layers of thermal insulation are considered as falling within the intent, scope and spirit of the present invention.

As described herein above, thermal pad 10 comprises outer and inner pouches 12 and 14 of pliant first and second material, respectively, and thermally transmitting material 32 includes a viscous material. As such, the shape of thermal pad 10 can be manually configured into different shapes for conformance with the shape and surface of the treatment area whereat it is positioned.

In one non-limiting example, thermal pad 10 is configured for administering thermal therapy to a woman's vaginal area and, as such, can be manipulated or adjusted to the shape of the vulvar area whereat it is positioned. Additionally, for administering thermal therapy to one or more pre-determined regions of the vaginal area, different quantities (or proportions) of thermally transmitting material 32 can be manually manipulated into and thereafter at least partially retained in the one or more areas defined by baffling mechanism 34. In such applications, thermal pad 10 can be formed to comfortably fit the vaginal area by, for example, providing a peanut-like shape comprising front region 56 defining front area 42, back region 58 defining back area 44, and central region 60 approximately equidistant between front and back regions 56 and 58. Accordingly, thermal pad 10 is configured such that when central region 60 is positioned approximately directly below the vaginal opening, front area 42 and back area 44 substantially cover the anterior and posterior regions of the vaginal area, and front region 56 and back region 58 respectively wrap around at least a portion of the front and at least a portion of the back of the woman's body.

In accordance with a non-limiting exemplary embodiment of the invention, exterior surface 18 of the second section of outer pouch 12 comprises one or more areas 62 having an increased coefficient of friction relative to other areas of exterior surface 18. For example, the areas of increased friction can be a rubberized surface. In this case, the one or more areas 62 can be configured for providing a substantially non-slip interface between thermal pad 10 and the woman's panties so that thermal pad 10 will be held in the desired position after the panty has been pulled fully on. Of course, shapes and sizes of the one or more areas 62 different from those illustrated in the figures are also possible. For example, a rubberized logo can be provided on the exterior surface 18 of the outer pouch 12. Other embodiments of thermal pad 10 include outer pouch 12 wherein the entirety of exterior surface 18 is configured to provide a substantially non-slip interface between thermal pad 10 and the woman's panties. All alternate configurations, as will become apparent to one skilled in the art, are considered as being within the bounds of the present invention.

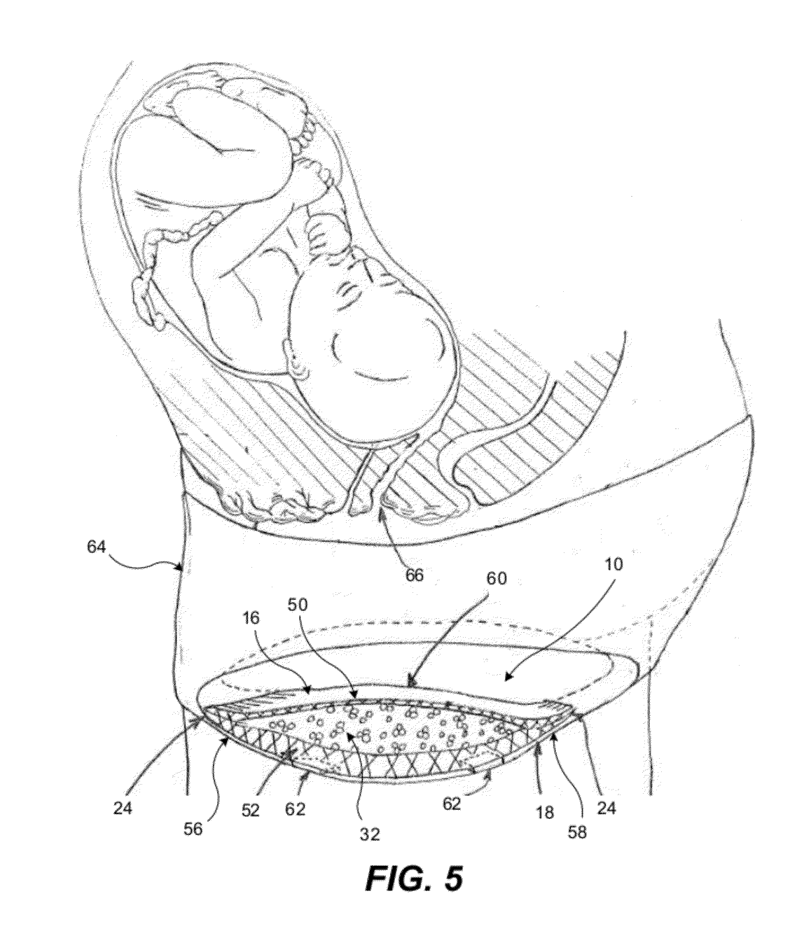

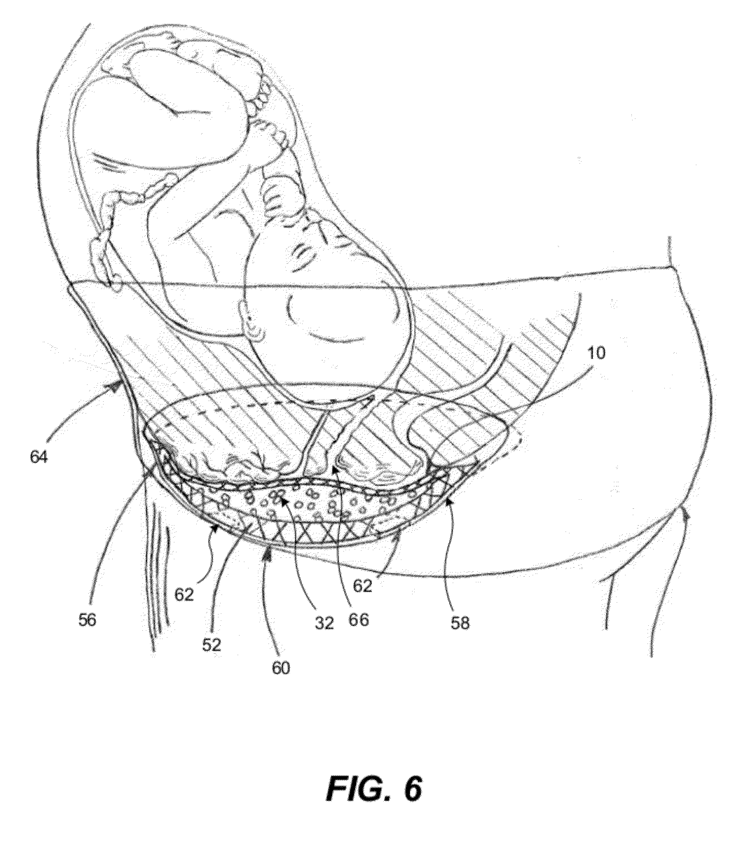

FIGS. 5 and 6 illustrate one particular method of using thermal pad 10 in accordance with a non-limiting exemplary embodiment of the invention. In particular, FIG. 5 shows a pre-positioning step wherein thermal pad 10 is positioned in the woman's panty 64 before being pulled fully-on as shown in FIG. 6. In accordance with an embodiment of the invention, thermal pad 10 is shown without the first and second gaps as described herein above with reference to FIGS. 3 and 4. In the interest of clarity, FIGS. 5 and 6 further show thermal pad 10 without baffling mechanism 34, though it should be appreciated that in some embodiments the thermal pad 10 may or may not include baffling mechanism 34.

In use, thermal pad 10 is manually manipulated to distribute, if desired, different quantities (or proportions) of thermally transmitting material 32 into the one or more areas within inner pouch 14. Either before or after the manual manipulation, thermal pad 10 is thermally conditioned, i.e., heated or cooled, to a desired temperature. Then, as depicted in FIG. 5, before panty 64 is pulled fully on, exterior surface 18 of outer pouch 12 is placed adjacent panty 64 with central region 60 approximately directly below vaginal opening 66 and exterior surface 16 of outer pouch 12 facing the vaginal area. As such, the one or more areas 62 in contact with panty 64 assist with retaining thermal pad 10 in the deployed position while panty 64 is not fully on, and more particularly after panty 64 has been pulled fully on. After thermal pad 10 has been appropriately positioned, such as shown in FIG. 5, panty 64 is pulled full on such that exterior surface 16 of outer pouch 12 contacts the woman's vaginal area as shown in FIG. 6. Thereafter, thermal pad 10 provides thermal therapy directly to, and in the vicinity of, the vaginal area in contact with exterior surface 16.

As shown in FIGS. 5 and 6, the varicose veins proximate the vaginal area, as well as the normal contours of the vaginal area provide an uneven and variable treatment surface. In some cases thermal pad 10 is first thermally conditioned and then placed in panty 64 with exterior surface 16 of outer pouch 12 positioned against the vaginal area as described herein above with reference to FIGS. 5 and 6. After placement, the pliant properties of the inner and outer pouch material, as well as the viscous nature of the thermally transmitting material 32 enable thermal pad 10 to self-configure itself, e.g., conform, to the contours of the vulvar region. This ability, especially in combination with the ability to manually manipulate the distribution of the thermally transmitting material about the baffling mechanism 34 provides a highly customizable thermal pad that is beneficial for providing thermal treatment to a wide variety of treatment surfaces, including highly individualized vaginal areas with varicose veins.

Thus, embodiments of the invention are disclosed. Although the present invention has been described in considerable detail with reference to certain disclosed embodiments, the disclosed embodiments are presented for purposes of illustration and not limitation and other embodiments of the invention are possible. One skilled in the art will appreciate that various changes, adaptations, and modifications may be made without departing from the spirit of the invention and the scope of the appended claims.

What is claimed is:

1. A thermal pad, comprising
a sealed, elongated outer pouch comprising a wall formed from an impermeable, pliant first material, the wall of the outer pouch comprising a first section having an exterior surface for applying to a treatment area and a second section having an exterior surface that faces away from the exterior surface of the first section;
a sealed, elongated inner pouch within the outer pouch, the inner pouch comprising a wall formed from an impermeable, pliant second material;
a viscous thermally transmitting material encased by the inner pouch for transmitting heat or cold to a user of the thermal pad; and
a baffling mechanism positioned within the inner pouch, the baffling mechanism dividing the interior of the inner pouch into at least two areas having fluid communication therebetween, the baffling mechanism configured to permit manual manipulation of the thermally transmitting material between the at least two areas and to at least partially retain the thermally transmitting material within one or more of the at least two areas subsequent to manual manipulation.

2. The thermal pad of claim 1, wherein the first and second sections of the outer pouch comprise opposing first and second layers of the first material sealed along at least a portion of a periphery of the outer pouch and wherein the inner pouch comprises opposing first and second layers of the second material, sealed along at least a portion of a periphery of the inner pouch.

3. The thermal pad of claim 1, further comprising a first layer of thermal insulation between the first section of the outer pouch wall and the inner pouch and a second layer of thermal insulation between the second section of the outer pouch wall and the inner pouch.

4. The thermal pad of claim 3, wherein a thermal resistance of the first layer of thermal insulation is less than a thermal resistance of the second layer of thermal insulation.

5. The thermal pad of claim 1, wherein the thermal pad is configured for administrating thermal therapy to a woman's vaginal area.

6. The thermal pad of claim 5, wherein the pliant first and second materials and the viscous thermally transmitting material enable the thermal pad to conform to a shape of a vulvar area whereat it is positioned.

7. The thermal pad of claim 5, wherein the thermal pad is configured to substantially cover an anterior, a central, and a posterior section of the vaginal area.

8. The thermal pad of claim 1, wherein the baffling mechanism comprises a plurality of baffles.

9. The thermal pad of claim 1, wherein the baffling mechanism comprises a longitudinal member positioned along a length of the interior of the elongated inner pouch and at least one lateral member positioned across the longitudinal member.

10. The thermal pad of claim 1, wherein the baffling mechanism is configured to restrict flow of the thermally transmitting material in at least two directions within the inner pouch.

11. The thermal pad of claim 1, wherein the baffling mechanism divides the interior of the inner pouch into four areas having fluid communication therebetween.

12. The thermal pad of claim 1, wherein the thermally transmitting material comprises a gelatinous material.

13. The thermal pad of claim 1, wherein at least a portion of the exterior surface of the second section of the outer pouch wall comprises a non-slip surface.

14. A thermal pad, comprising:
a sealed, elongated outer pouch comprising a wall formed from an impermeable, pliant first material, the wall of the outer pouch comprising a first section having an exterior surface for applying to a treatment area and a second section having an exterior surface that faces away from the exterior surface of the first section;
a sealed, elongated inner pouch within the outer pouch, the inner pouch comprising a wall formed from an impermeable, pliant second material;
a viscous thermally transmitting material encased by the inner pouch for transmitting heat or cold to a user of the thermal pad;
a first layer of thermal insulation between the first section of the outer pouch wall and the inner pouch; and
a second layer of thermal insulation between the second section of the outer pouch wall and the inner pouch.

15. The thermal pad of claim 14, wherein the first and second sections of the outer pouch wall comprise opposing first and second layers of the first material sealed along at least a portion of a periphery of the outer pouch and wherein the inner pouch comprises opposing first and second layers of the second material, sealed along at least a portion of a periphery of the inner pouch.

16. The thermal pad of claim 15, wherein the first and second layers of the first material are separate sheets having respective perimeters sealed together to form the outer pouch, and wherein the first and second layers of the second material are separate sheets having respective perimeters sealed together to form the inner pouch.

17. The thermal pad of claim 16, wherein the first layer of thermal insulation has a first thermal resistance and the second layer of thermal insulation has a second thermal resistance, wherein the second thermal resistance is greater than the first thermal resistance so as to provide greater heat transfer between the thermally transmitting material and the treatment area than between the thermally transmitting material and an external environment adjacent the exterior surface of the second section of the outer pouch wall.

18. The thermal pad of claim 14, wherein the thermal pad is configured for administrating thermal therapy to a woman's vaginal area.

19. The thermal pad of claim 18, wherein the pliant first and second materials and the viscous thermally transmitting material enable the thermal pad to conform to a shape of a vulvar area whereat it is positioned.

20. A method for administering thermal therapy to a woman's vaginal area, comprising:
configuring a thermal pad in a first treatment configuration, the thermal pad comprising a sealed elongated outer pouch, a sealed elongated inner pouch within the outer pouch, a viscous thermally transmitting material encased by the inner pouch, and a baffling mechanism positioned within the inner pouch, the configuring comprising manipulating the thermal pad to direct the viscous thermally transmitting material about the baffling mechanism in a desired distribution;
cooling the thermally transmitting material within the thermal pad; and
positioning the thermal pad adjacent to a woman's vaginal area to allow heat transfer between the vaginal area and the thermally transmitting material.
